(19) United States
(12) Patent Application Publication
(10) Pub. No.: US 2017/0311807 A1
(43) Pub. Date: Nov. 2, 2017

(54) APPARATUSES FOR DETECTING BIOMARKERS AND METHODS FOR USING THE SAME

(71) Applicant: HILL-ROM SERVICES, INC., BATESVILLE, IN (US)

(72) Inventors: Yongji Fu, Harrison, OH (US); Charles A. Lachenbruch, Batesville, IN (US)

(73) Assignee: HILL-ROM SERVICES, INC., BATESVILLE, IN (US)

(21) Appl. No.: 15/496,484

(22) Filed: Apr. 25, 2017

Related U.S. Application Data
(60) Provisional application No. 62/328,154, filed on Apr. 27, 2016.

(51) Int. Cl. A61B 5/00; A61B 5/053

(52) U.S. Cl. CPC A61B 5/0084; A61B 5/742; A61B 5/7405; A61B 5/0538; A61B 5/441; A61B 5/6848

(57) ABSTRACT

Apparatus, methods, and systems for detecting biomarkers are disclosed. A sensing apparatus for detecting the presence of at least one biomarker in subcutaneous tissue is disclosed, the sensing apparatus including a probe comprising a shaft, and a biomarker detection material coated on at least a portion of the shaft of the probe. The sensing apparatus further includes a sensor communicatively coupled to the biomarker detection material, the sensor configured to detect a change in at least one of an electrical property and an optical property of the biomarker detection material. When a change in the at least one of an electrical property and optical property of the biomarker detection material is detected, the change is indicative of the presence of at least one biomarker.

### CROSS REFERENCE TO RELATED APPLICATIONS

The present application claims priority to U.S. Provisional Patent Application Ser. No. 62/328,154 filed Apr. 27, 2016 and entitled “APPARATUSES FOR DETECTING BIOMARKERS AND METHODS FOR USING THE SAME,” the entirety of which is incorporated by reference herein.

### TECHNICAL FIELD

The present specification generally relates to apparatuses for detecting biomarkers and, more specifically to apparatuses which include a probe which may be inserted into subcutaneous tissue to detect the presence of biomarkers and methods for using the same.

### BACKGROUND

A pressure ulcer is a localized injury to the skin or an underlying tissue arising from sustained pressure or pressure in combination with shear force and/or friction. Pressure ulcers may develop on the surface of the skin or present deep within the tissue. When pressure ulcers develop deep within the tissue, there may be no visible indication of the development of the pressure ulcer for a long period of time, making early detection a challenge.

Presently, in order to detect the onset of pressure ulcers within the tissue, blood samples are drawn, and blood plasma is screened for the presence of such biomarkers. However, screening for biomarkers in this way can be excessively time consuming and labor intensive as blood samples would need to be drawn intermittently and screening would need to be separately performed for each sample.

Accordingly, there is a need for alternative apparatuses and methods to facilitate early and continuous detection of the formation of pressure ulcers within tissues.

### SUMMARY

In one embodiment, a sensing apparatus for detecting a development of a pressure ulcer in a subcutaneous tissue may include a probe and a biomarker detection material coated on at least a portion of the probe. An electrical property or an optical property of the biomarker detection material may change upon exposure to a pressure ulcer biomarker. A sensor may be coupled to the biomarker detection material. The sensor may be configured to detect the electrical property or the optical property of the biomarker detection material. A detected change in the electrical property or the optical property of the biomarker detection material may be indicative of the development of the pressure ulcer.

In another embodiment, a method for detecting a presence of at least one biomarker in a subcutaneous tissue may include positioning a probe of a probe unit into the subcutaneous tissue. The probe may include a biomarker detection material coated on at least a portion of the probe wherein an optical property or an electrical property of the biomarker detection material changes upon exposure to a pressure ulcer biomarker. Thereafter, the optical property or the electrical property of the biomarker detection material may be detected and a change in the optical property or the electrical property of the biomarker detection material may be determined. A warning signal may be provided when the change in the optical property or the electrical property of the biomarker detection material is indicative of a presence of the pressure ulcer biomarker.

In yet another embodiment, a sensing apparatus for detecting a development of a pressure ulcer in a subcutaneous tissue may include a probe unit comprising a probe having a biomarker detection material coated on at least a portion of the probe. An optical property of the biomarker detection material may change upon exposure to the pressure ulcer biomarker. The sensing apparatus may also include a detection unit comprising a sensor and a light source emitting a reference light. At least one optical fiber may optically couple the light source to the biomarker detection material and the sensor to the biomarker detection material. The sensor may be configured to detect an optical property of the biomarker detection material, a change in the optical property of the biomarker detection material being indicative of the development of the pressure ulcer.

These and additional features provided by the embodiments of the present disclosure will be more fully understood in view of the following detailed description, in conjunction with the drawings.

### BRIEF DESCRIPTION OF THE DRAWINGS

The embodiments set forth in the drawings are illustrative and exemplary in nature and not intended to limit the disclosure. The following detailed description of the illustrative embodiments can be understood when read in conjunction with the following drawings, where like structure is indicated with like reference numerals and in which:

FIG. 1 schematically depicts a sensing apparatus for detecting the presence of at least one biomarker in subcutaneous tissue according to one or more embodiments shown and described herein;

FIG. 2 schematically depicts a probe unit of the sensing apparatus according to one or more embodiments shown and described herein;

FIG. 3 schematically depicts a cross section of an optical fiber for use with one or more embodiments of the sensing apparatus shown and described herein;

FIG. 4 schematically depicts the probe unit inserted into the subcutaneous tissue of a subject according to one or more embodiments shown and described herein;

FIG. 5 schematically depicts a detection unit of the sensing apparatus according to one or more embodiments shown and described herein;

FIG. 6 schematically depicts one embodiment of a probe unit with the detection unit attached thereto according to one or more embodiments shown and described herein;

FIG. 7 schematically depicts a receiving unit of the sensing apparatus according to one or more embodiments shown and described herein;

FIG. 8 graphically depicts a flowchart of a method for detecting the presence of at least one biomarker in a subcutaneous tissue according to one or more embodiments shown and described herein;

FIG. 9 schematically depicts an alternative embodiment of a sensing apparatus for detecting and determining a characteristic of at least one biomarker in subcutaneous tissue according to one or more embodiments shown and described herein; and

FIG. 10 schematically depicts an alternative embodiment of a sensing apparatus for detecting and determining a characteristic of at least one biomarker in the subcutaneous tissue according to one or more embodiments shown and described herein.

### DETAILED DESCRIPTION

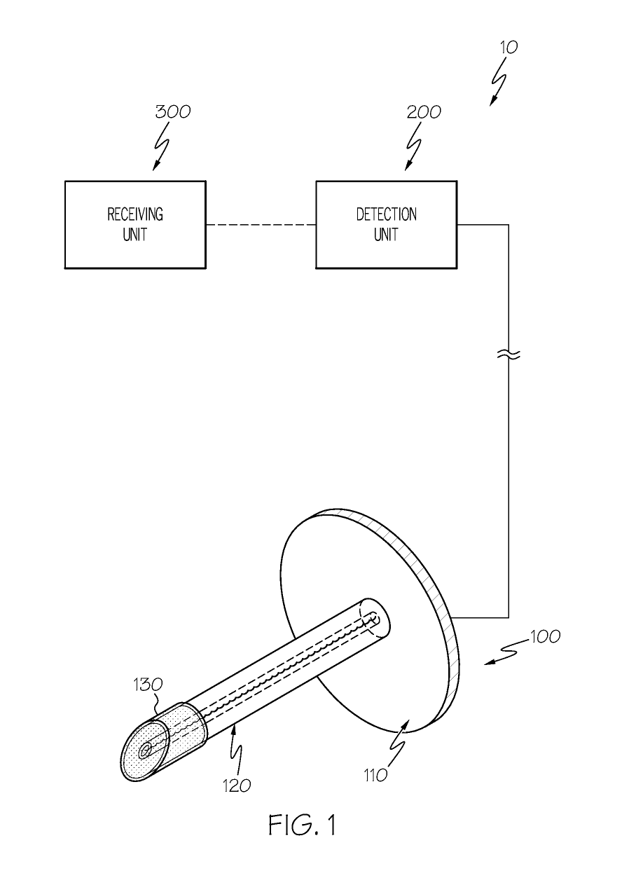

Embodiments disclosed herein generally relate to apparatuses for detecting biomarkers in subcutaneous tissue and methods for using the same. Referring generally to FIG. 1, a sensing apparatus for detecting the presence of biomarkers in a subcutaneous tissue of a subject is shown. The sensing apparatus may include a probe and a biomarker detection material coated on at least a portion of the probe. An electrical property or an optical property of the biomarker detection material may change upon exposure to a pressure ulcer biomarker. A sensor may be coupled to the biomarker detection material. The sensor may be configured to detect the electrical property or the optical property of the biomarker detection material. A detected change in the electrical property or the optical property of the biomarker detection material may be indicative of the development of the pressure ulcer. Various embodiments of apparatuses for detecting biomarkers, and methods for using the same, will be described herein with specific reference to the appended drawings.

The presence of certain biomarkers in the subcutaneous tissue of a subject has been found to be indicative of the formation of pressure ulcers in the tissue. As a pressure ulcer begins to form, cells in the area of the pressure ulcer release these biomarkers into the interstitial fluid of the subcutaneous tissue. By detecting the presence of certain biomarkers in the interstitial fluid of the subcutaneous tissue contemporaneously with the release of the biomarkers, early detection of deep tissue pressure ulcers is possible in real time, allowing users to take immediate action to mitigate the formation of the pressure ulcers in subjects.

As used herein, a “user” may be a health care professional such as a doctor, a nurse, or another caregiver. In some embodiments, the user may be an individual tasked with updating medical records of a subject.

As used herein, a “subject” may be an individual who is being monitored for the presence of the at least one biomarker. In embodiments, the subject may be a patient, such as a patient in a medical care facility including, without limitation, a hospital, a surgical center, a nursing home, or a long-term care facility.

As used herein, a “biomarker” is a biological substance that may be used as an indicator of a state or condition of the tissue(s) in which it is present. These biomarkers are used to assess the susceptibility and severity of medical conditions, such as, for example pressure ulcers.

As used herein, a “pressure ulcer biomarker” is a biomarker indicative of the development of a pressure ulcer in the tissue of a subject. The pressure ulcer biomarker may include proteomic biomarkers including, without limitation, creatine kinase, myoglobin, H-FABP, troponin, myosin, c-reactive protein, creatinine phosphokinase, interlunkin 1-α, and/or combinations thereof.

Referring to FIG. 1, one embodiment of a sensing apparatus 10 is schematically depicted. The sensing apparatus 10 may be used to detect the presence of a biomarker in subcutaneous tissue of a subject which may be indicative of the development of a pressure ulcer in the subcutaneous tissue of a subject. In embodiments, the sensing apparatus 10 generally includes a probe unit 100, a detection unit 200, and a receiving unit 300. The probe unit 100 may include a probe 120 and a mounting member 110 that assists in affixing the probe 120 to a subject. The probe unit 100 is communicatively coupled to the detection unit 200. The detection unit 200 is in turn communicatively coupled to the receiving unit 300. The phrase “communicatively coupled,” as used herein, refers to the ability of the recited components to send and/or receive signals, including optical signals, electrical signals, electromagnetic signals, and the like. The recited components may be communicatively coupled by wires, optical fibers, or the like. Alternatively, the recited components may be communicatively coupled wirelessly using radio frequency (RF) transmitters, receivers, and/or transceivers.

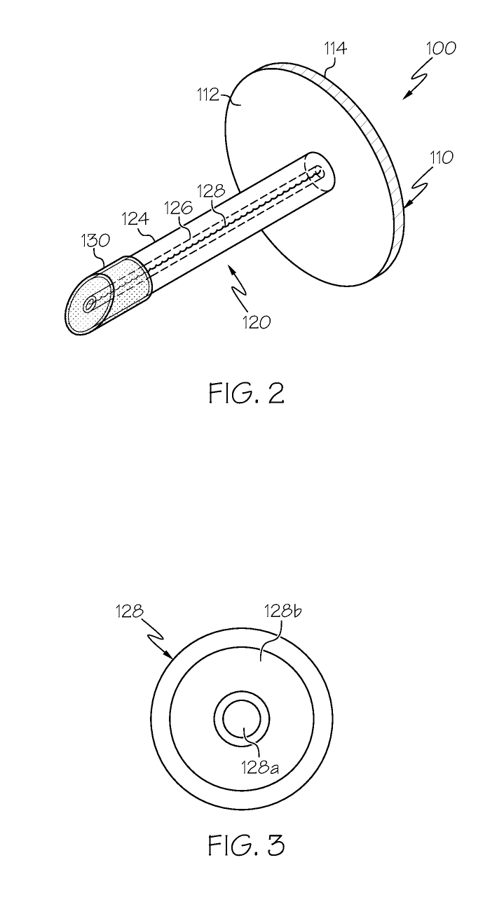

Referring now to FIGS. 1 and 2, FIG. 2 schematically depicts the probe unit 100 of the sensing apparatus 10 of FIG. 1. In the embodiment of the probe unit 100 depicted in FIGS. 1 and 2, the probe unit 100 generally includes a mounting member 110 and a probe 120 at least a portion of which is coated with a biomarker detection material 130. While FIGS. 1 and 2 schematically depict the probe unit 100 as including a mounting member 110, it should be understood that the mounting member 110 is optional and that, in some embodiments, the probe unit 100 may be constructed without the mounting member 110.

In embodiments of the probe unit 100 which include a mounting member 110, the mounting member 110 may be, for example, a flexible substrate or sheet of material that facilitates affixing the probe unit 100 to the skin of a subject. More specifically, the mounting member 110 may be sufficiently pliable to facilitate forming the mounting member 110 to complement the contours of the specific location of the subject to which the probe unit 100 is affixed. In certain embodiments, the mounting member 110 may be made of, for example, cloth, polymeric materials or a combination of cloth and polymeric materials. The cloth may include natural and/or synthetic fibers and may be a woven material or non-woven material. In embodiments, the materials from which the mounting member 110 is constructed are medical grade materials suitable for use in a clinical setting.

In certain embodiments, the mounting member 110 has a proximal surface 112 which is positioned on the skin of the subject when the probe unit is placed on the subject. In embodiments, the proximal surface 112 is coated with an adhesive to assist in affixing the probe unit 100 to the skin of a subject. The adhesive may be a medical grade adhesive such as, for example, the adhesives used in adhesive-type bandages. The proximal surface 112 of the mounting member 110 may also be coated with local anesthetics (such as lidocaine or the like) to suppress discomfort caused by the insertion of the probe 120 in the subcutaneous tissue of the subject. The mounting member 110 may also include a distal surface 114 opposite the proximal surface 112 of the mounting member 110. The distal surface 114 may serve as a mounting surface for other components of the probe unit 100, as will be described in further detail herein.

As shown in FIG. 2, the probe unit 100 further includes the probe 120. In embodiments where the probe unit 100 includes a mounting member 110, the probe 120 extends from the proximal surface 112 of the mounting member 110. In some embodiments, the probe 120 is perpendicular to the proximal surface 112 of the mounting member 110 when the mounting member 110 is in a planar state (i.e., when the mounting member is not contoured or otherwise deformed). In other embodiments, the probe 120 may be angled with respect to the proximal surface 112 such that the smallest angle between the probe 120 and the proximal surface 112 is less than 90 degrees when the mounting member 110 is in a planar state.

In embodiments, the probe 120 is a slender, pointed instrument suitable for puncturing skin and penetrating into the underlying tissues. For example, the probe 120 may be a needle or a micro needle having a similar configuration as a hypodermic needle. The probe 120 may be made of materials suitable for medical applications such as plastic, surgical steel or the like. In embodiments, the outer wall of the probe 120 defines a shaft 124 which includes a lumen 126 extending there through. In certain embodiments, optical fiber(s) or electrical wire(s) may extend through the lumen 126 of the shaft 124. The optical fiber(s) or electrical wire(s) may be used to send and/or receive signals, such as data signals, relating to the presence of at least one biomarker in the interstitial fluid of the subcutaneous tissue in which the probe 120 is inserted. While the probe 120 has been shown and described herein as containing a shaft 124 through which the lumen 126 extends, it should be understood that, in other embodiments (not shown), the shaft 124 may be formed without a lumen 126, such as when the shaft 124 is solid. In these embodiments, the optical fiber(s) and/or electrical wire(s) associated with the probe unit may be disposed on an exterior surface of the shaft 124, such as in a groove or channel formed on the exterior surface of the shaft 124.

Still referring to FIG. 2, the probe unit 100 further includes the biomarker detection material 130. The biomarker detection material 130 may be coated on at least a portion of the shaft 124 of the probe 120. In embodiments, the biomarker detection material 130 may also be coated on and in at least a portion of the lumen 126 of the probe 120. The biomarker detection material 130 is selected such that, when the biomarker detection material 130 comes into contact with a specific biomarker, an optical or electrical property of the biomarker detection material 130 changes, thereby indicating the presence of the biomarker. For example, in the embodiments described herein, the biomarker detection material 130 is suitable for detecting the presence of a biomarker indicative of the development of a pressure ulcer. Such biomarkers include, without limitation, creatin kinase, myoglobin, H-FABP, troponin, myosin, C-reactive protein, creatinine phosphokinase, interlunkin 1-α and/or combinations thereof. By way of example and without limitation, when the biomarker detection material 130 comes into contact with a specific biomarker, an optical property of the biomarker detection material may change and the change in this property may be detected and used to determine the presence of the biomarker. Similarly, when the biomarker detection material comes into contact with a specific biomarker, an electrical property of the biomarker detection material may change and the change in this property may be detected and used to determine the presence of the biomarker.

In embodiments, the biomarker detection material 130 may include a material or chemical compound which reacts with one or more of the aforementioned biomarkers (i.e., the reactive material or reactive chemical compound). For example and without limitation, the reactive material or reactive chemical compound may include one or more components from an assay for the detection of pressure ulcer biomarkers. Suitable assays include, without limitation, an ELISA assay, an immuno-nephelometry assay, an immunoturbidimetry assay, rapid-enzyme immunoassays, an immunoenzymometric assay, an immunoradiometric assay, and/or combinations thereof. For example and without limitation, a biomarker detection material suitable for detecting the creatine kinase biomarker may include the Creatine Kinase Activity Assay Kit (Colorimetric) sold by Abcam plc under product number ab155901 and/or one or more components thereof such as the CK Enzyme Mix. A biomarker detection material suitable for detecting the troponin biomarker may include the Enzymun-Test Troponin T from Roche Diagnostics and/or one or more components thereof. A biomarker detection material suitable for detecting the Interlukin 1-α biomarker may include the IL-1α (Interleukin-1 alpha) Human ELISA Kit sold by Abcam plc under product number ab100560 and/or one or more components thereof. In embodiments, the biomarker detection material 130 may include a combination of different materials each of which may be suitable for detecting a different biomarker.

In embodiments, the biomarker detection material 130 may further include a carrier material to facilitate coating on the probe 120. For example, the carrier material may include a hydrogel in which the reactive material or reactive chemical compound is entrained. The mixture of the hydrogel and the reactive material or reactive chemical compound may be applied to the probe 120 as a coating. Alternatively, the reactive material or reactive chemical compound may be applied to the probe 120 and the hydrogel may be applied over the reactive material or reactive chemical compound such that the biomarker detection material 130 is a multi-layer coating on the probe 120.

In embodiments, the probe 120 may further include electrical wire(s) and/or optical fiber(s) coupled to the biomarker detection material 130 to facilitate detection of a change in an optical or electrical property of the biomarker detection material 130 due to the presence of a biomarker. For example, in one embodiment, the probe 120 includes at least one optical fiber 128 that extends through the lumen 126 of the probe 120, as depicted in FIG. 2. The at least one optical fiber 128 may be formed from silica-based glass or, alternatively, a polymeric material. A cross section of a suitable optical fiber is schematically depicted in FIG. 3. The at least one optical fiber 128 may include an inner core 128a, and an outer core 128b, the outer core 128b surrounding the inner core 128a. The at least one optical fiber 128 is configured to carry light to and from the biomarker detection material 130. Accordingly, it should be understood that the at least one optical fiber 128 is optically coupled to the biomarker detection material 130. Additionally, the at least one optical fiber 128 may be optically coupled to a sensor 210 (shown in FIG. 5) and a light source 230 (also shown in FIG. 5), as will be described in further detail herein. In embodiments, a reference light is directed from the light source 230 through the at least one optical fiber 128 onto the biomarker detection material 130 and reflected light is collected from the biomarker detection material 130 by the at least one optical fiber 128 and directed into the sensor 210. The reference light (also denoted herein as R_{L}) is light directed onto the biomarker detection material 130. The reflected light (also denoted herein as R_{R}) is light collected from the biomarker detection material 130. In embodiments, the reference light may be directed onto the biomarker detection material 130 via the inner core 128a of the at least one optical fiber 128, and the reflected light may be collected from the biomarker detection material via the outer core 128b of the at least one optical fiber 128. Alternatively, the reference light may be directed onto the biomarker detection material 130 via the outer core 128b of the at least one optical fiber 128, and the reflected light may be collected from the biomarker detection material via the inner core 128a of the at least one optical fiber 128. In other embodiments, two optical fibers may be used within the probe 120, one to direct the reference light onto the biomarker detection material 130, and the other to collect the reflected light from the biomarker detection material 130.

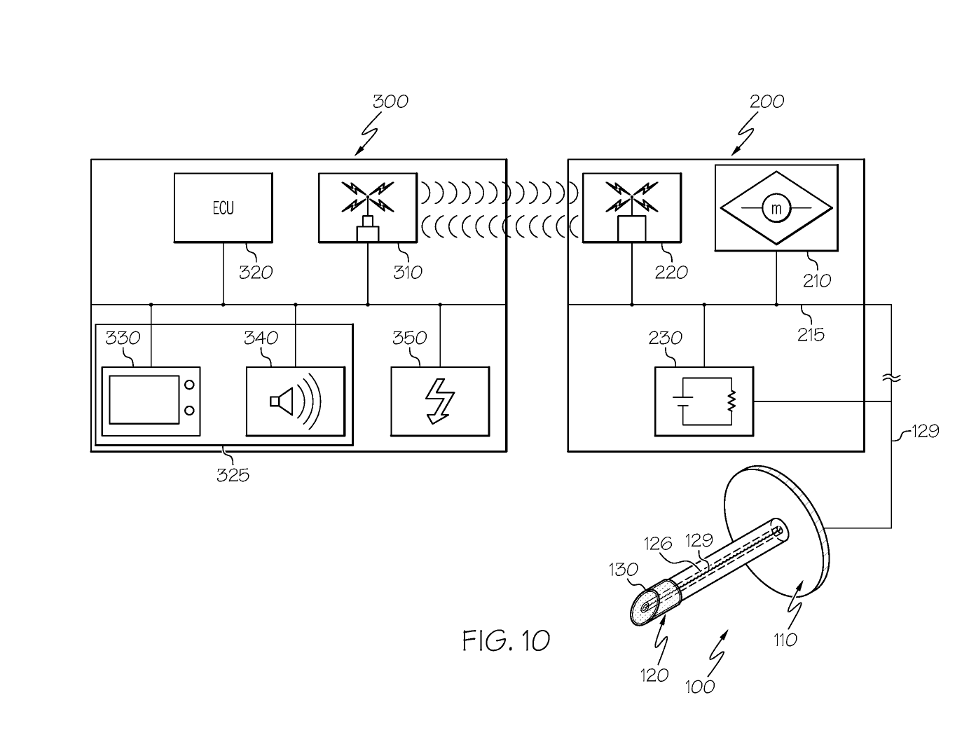

In embodiments, the at least one optical fiber 128 is employed when the sensing apparatus 10 is used to detect the presence of the at least one biomarker based on an optical property of the biomarker detection material. In embodiments where the sensing apparatus 10 is used to detect the presence of the at least one biomarker based on an electrical property of the biomarker detection material, electrical wire(s) 129 (shown in FIG. 10) may be used instead of optical fiber. In embodiments, the electrical wire(s) 129 are communicatively coupled to the biomarker detection material 130, such as by direct contact. That is, the electrical wire(s) 129 may be embedded in the biomarker detection material 130 or the biomarker detection material may be at least partially coated on the electrical wire(s) 129. In these embodiments, the electrical wire(s) 129 are also communicatively coupled to the sensor 210.

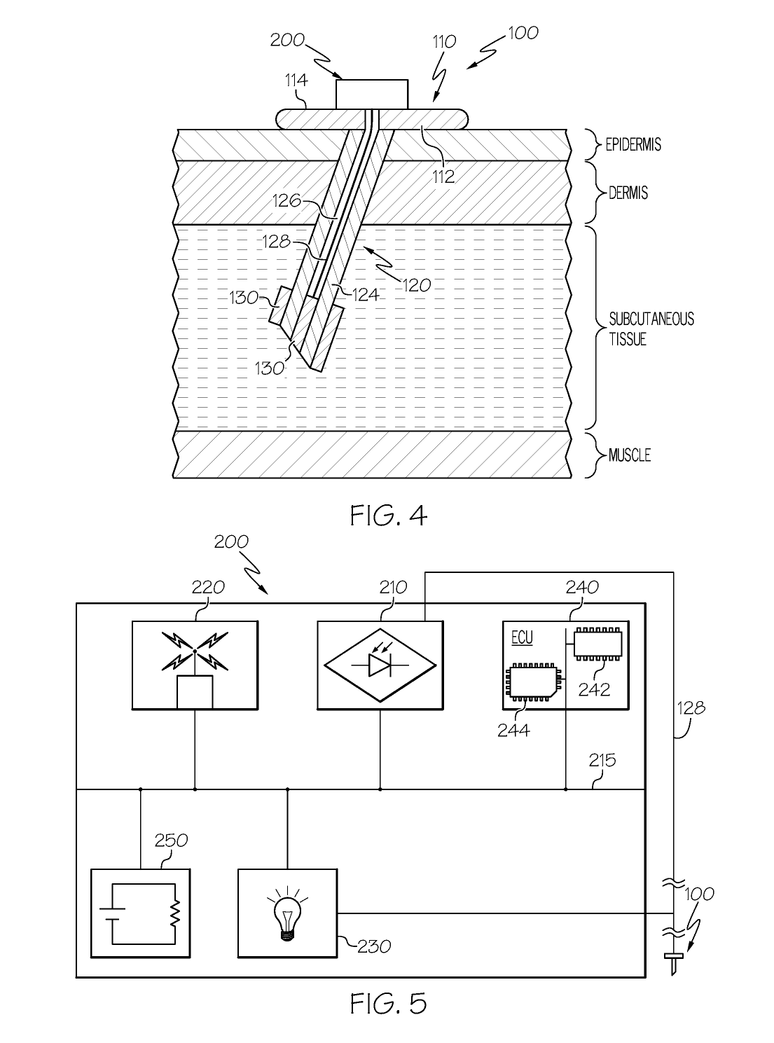

Referring now to FIG. 4, the probe 120 of the probe unit 100 is shown inserted into the subcutaneous tissue of a subject. The proximal surface 112 of the mounting member 110 is used to affix the probe unit 100 onto the subject and hold the probe 120 in place. The probe 120 is of sufficient length to penetrate the epidermis and the dermis regions and reach the subcutaneous tissue of the subject. In the embodiment shown in FIG. 4, the probe 120 includes the shaft 124 and the lumen 126, the lumen 126 containing the at least one optical fiber 128. The at least one optical fiber 128 extends through the lumen 126 of the probe 120 and into the mounting member 110 and connects to the detection unit 200. Further, the shaft 124 of the probe 120 is coated with the biomarker detection material 130, and the at least one optical fiber 128 is positioned to emit the reference light onto the biomarker detection material 130 and collect reflected light from the biomarker detection material 130.

Referring now to FIG. 5, one embodiment of a detection unit 200 of the sensing apparatus 10 is schematically depicted. The detection unit 200 generally includes a sensor 210, a transmitter 220, a light source 230, an electronic control unit (ECU) 240, and a power source 250. The sensor 210, transmitter 220, light source 230 and power source 250 (e.g., a DC power source such as a battery) are coupled to one another via the communication pathway 215 and/or the ECU 240. The various components of the detection unit 200 are described below in further detail.

In the embodiments described herein, the communication pathway 215 may be formed from any medium suitable for transmitting electrical signals and/or optical signals, such as for example, conductive wires, conductive traces, optical waveguides, or the like. In one exemplary embodiment, the sensor 210, transmitter 220, light source 230, and power source 250 are positioned on an electronic substrate, such as a silicon wafer or the like, and the communication pathway 215 is a series of electrical traces interconnecting the various components of the detection unit 200.

As depicted in FIG. 5, the detection unit 200 includes an ECU 240. The ECU 240 is electrically coupled to the power source 250 which supplies power to the ECU 240 and related components of the ECU 240. The ECU 240 may include a processor 244 for executing machine readable and executable instructions and a non-transitory electronic memory 242 for storing the machine readable and executable instructions. In embodiments, the processor 244 may be an integrated circuit, microchip, computer, or any other computing device capable of executing machine readable and executable instructions. The electronic memory 242 may be RAM, ROM, flash memory, a hard drive, or any other form of non-transitory memory capable of storing machine readable and executable instructions. In the embodiments described herein, the processor 244 and the electronic memory 242 are integral with the ECU 240. However, it is noted that, in alternative embodiments, the ECU 240, the processor 244, and the electronic memory 242 may include a series of discrete components in electrical communication with one another. The machine readable and executable instructions stored in the electronic memory 242 of the ECU 240 facilitate the operation of the detection unit 200 and the probe unit 100 including, without limitation, the collection of data (i.e., optical or electrical signals) from the probe unit 100 by the detection unit 200 and the transmission of that data to the receiving unit 300.

In embodiments, the ECU 240 may be configured to receive electrical signals from the sensor 210 via the communication pathway 215. In embodiments, the ECU 240 may be configured to determine a biomarker detection signal indicating the presence (or absence) of a biomarker based on the electrical signals received from the sensor 210 and transmit the biomarker detection signal with the transmitter 220. The ECU 240 may also be configured to control the light source 230 and the transmitter 220.

In the embodiment of the detection unit 200 depicted in FIG. 5, the detection unit 200 is configured to detect an optical property of the biomarker detection material 130 of the probe unit 100. To facilitate the detection of an optical property of the biomarker detection material 130, the detection unit 200 includes a light source 230. The light source 230 is optically coupled to the at least one optical fiber 128 which, in turn, is optically coupled to the biomarker detection material 130 of the probe unit 100, as described herein. The light source 230 may be configured to send reference light of a predetermined wavelength and/or intensity via the at least one optical fiber 128 to the probe unit 100 such that the reference light is incident on the biomarker detection material 130. The light source 230 may be selected such that the optical property (such as wavelength and/or intensity) of the reference light emitted by the light source 230 is suitable for detecting a change in the optical properties of the biomarker detection material 130. That is, the light source 230 may be selected to emit a certain wavelength and/or intensity of light depending on the type of biomarker detection material being used and the biomarkers being detected by the probe unit 100 in the subcutaneous tissue such that a change in the wavelength and/or intensity of the light is detectable with the sensor 210. In embodiments, the light source 230 may be a broad-band light source (e.g., an incandescent light source, an electric discharge light source, or a white light source) or a coherent light source. Examples of electric discharge sources include arc lamps, fluorescent lamps, carbon lamps, mercury-vapor lamps, metal-halide lamps, and the like. Examples of incandescent sources include halogen lamps, incandescent light bulbs, and the like. Examples of coherent light sources include light-emitting diodes (LED).

In embodiments, the light source 230 is electrically coupled to the power source 250 which provides electrical energy to the light source 230. In some embodiments, the light source 230 may also be electrically coupled to the ECU 240 which may facilitate actuating the light source 230 (i.e., switching the light source on and off) at predetermined intervals.

As noted above, the detection unit 200 includes a sensor 210. The sensor 210 may be communicatively coupled to the other components in the detection unit 200 via the communication pathway 215. For example, in embodiments, the sensor 210 is communicatively coupled to the ECU 240 via the communication pathway 215 and to the transmitter 220 via the ECU 240 and the communication pathway 215. The sensor 210 is also coupled to the power source 250 which provides electrical energy to the sensor 210. Additionally, the sensor 210 is communicatively coupled to the biomarker detection material 130 with the at least one optical fiber 128, as depicted in FIGS. 4 and 5. In these embodiments, the sensor 210 is used to detect an optical property of the biomarker detection material 130.

In embodiments where the sensor 210 is configured to detect a change in an optical property of the biomarker detection material 130, the sensor 210 may be an optical detector, such as a photodetector or a photodiode. In embodiments where the sensor 210 is an optical detector, the optical detector may be configured to detect a wavelength (or a change in wavelength) of the reflected light collected from the biomarker detection material. Additionally, in some embodiments, the sensor 210 may be an optical detector configured to detect an intensity (or a change in intensity) of the reflected light collected from the biomarker detection material 130.

As noted hereinabove, in some embodiments, a change in an electrical property of the biomarker detection material 130 may be used to detect the presence of a biomarker. Accordingly, in alternative embodiments (such as the embodiment of the sensing apparatus depicted in FIG. 10), the sensor 210 is configured to detect a change in an electrical property of the biomarker detection material 130. In these embodiments, the detection unit 200 does not include light source 230. Instead, in these embodiments, the sensor 210 is communicatively coupled to the probe unit 100 (specifically the biomarker detection material 130 of the probe unit 100) with electrical wire(s) 129 (FIG. 10), as described herein. In these embodiments, the sensor 210 may be configured to detect one or more of capacitance, impedance, resistance, and/or voltage of the biomarker detection material 130, or a change thereof. That is, the sensor 210 in this embodiment may be a multi-meter, a capacitance meter, an ammeter, an ohmmeter, or a voltmeter. In these embodiments, the power source 235 is communicatively coupled to the sensor 210 and to the biomarker detection material 130 with the electrical wire(s). In embodiments, the power source 235, working in conjunction with the ECU 240, is configured to provide a reference electrical signal to the biomarker detection material 130 and the sensor 210 is configured to detect an electrical property of the biomarker detection material 130 based on the reference electrical signal and/or a returned electrical signal. The sensor 210 transmits an electrical signal indicative of the detected electrical property to the ECU 240 which, in turn, determines a biomarker detection signal based on this electrical signal. In certain embodiments, electrical characteristics of the reference electrical signal may be selected depending on the biomarker detection material 130 and the biomarker being detected.

In embodiments where the sensor 210 is configured to detect an optical property of the biomarker detection material, the sensor 210 outputs an electrical signal to the ECU 240 indicative of the detected optical property of the biomarker detection material 130. In embodiments, the ECU 240 of the detection unit is configured to compare the optical property of the reflected light with the reference light. Based on the comparison, the ECU 240 is configured to determine a biomarker detection signal indicative of the presence or absence of a biomarker based on a change of an optical property (such as a change in the wavelength and/or intensity of the reflected light from the biomarker detection material relative to the reference light) of the biomarker detection material 130. In these embodiments, a change in the optical property is indicative of the presence of a biomarker in the interstitial fluid of the subcutaneous tissue in which the probe 120 is inserted.

In alternative embodiments, the ECU 240 of the detection unit 200 receives the electrical signal from the sensor 210 and determines a biomarker detection signal which may be further compared and analyzed relative to the reference light by the receiving unit 300 (FIG. 1). In this embodiment, the biomarker detection signal is indicative of the detected optical property of the biomarker detection material and further processing of the biomarker detection signal is performed by the receiving unit 300 to determine the presence or absence of a biomarker in the interstitial fluid of the subcutaneous in which the probe 120 is inserted.

As shown in FIG. 5, the detection unit 200 further includes a transmitter 220. The transmitter 220 may be communicatively coupled to the ECU 240, as described herein above. The transmitter 220 is also communicatively coupled to the receiving unit 300 of the sensing apparatus 10 (FIG. 1). In embodiments, the transmitter 220 is communicatively coupled to the receiving unit 300 wirelessly, such as when the transmitter 220 is an RF transmitter using Bluetooth® communication protocols, IEEE 802.11 wireless communication protocols, near-field communication protocols, or any other communication protocol suitable for facilitating radio frequency communications between electronic devices. Alternatively, the transmitter 220 may be directly coupled to the receiving unit 300, such a by wires and/or optical fiber.

In embodiments, the transmitter 220 is configured to transmit the biomarker detection signal from the detection unit 200 to the receiving unit 300 (shown in FIG. 1). In embodiments, the transmitter 220 may also be configured to receive data and/or instructions from, for example, the receiving unit 300. Accordingly, it should be understood that, in embodiments, the transmitter 220 may be a transceiver suitable for both sending and receiving data signals.

In an alternative embodiment (not depicted), the detection unit 200 may further include a warning indicator (not shown) such as a visual and/or audible warning indicator. The warning indicator may be communicatively coupled to the ECU 240 such that, when the electrical signal received from the sensor 210 indicates the presence of a biomarker (as determined by the ECU 240), the ECU 240 activates the warning indicator to provide a visible and/or audible indication of the presence of the biomarker. The warning indicator may be used in conjunction with the transmission of the biomarker detection signal to the receiving unit 300 or as a substitute therefore.

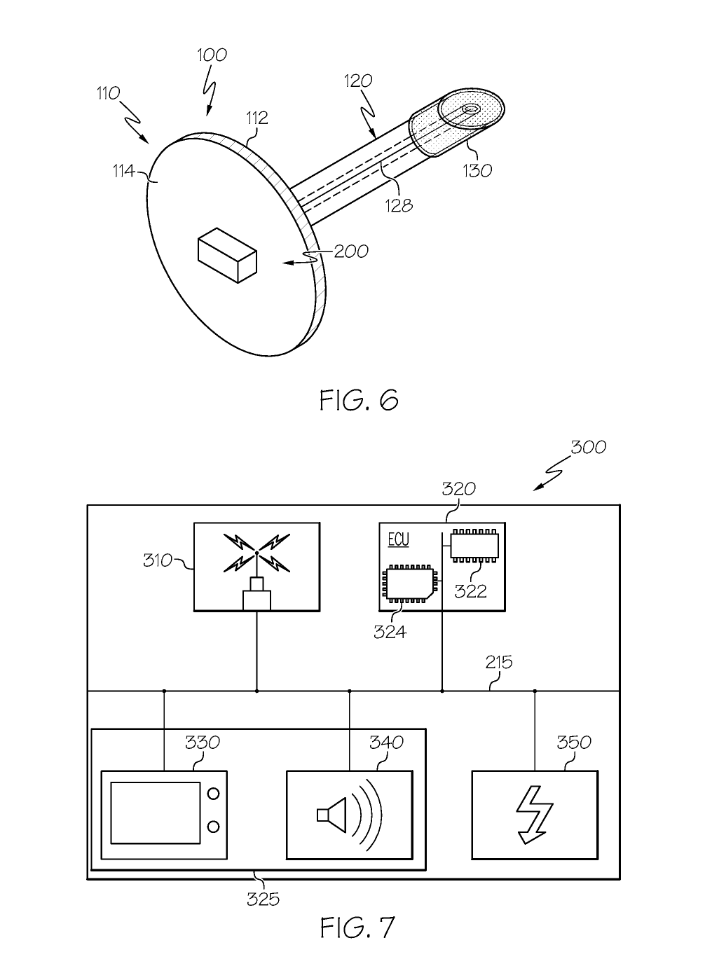

Referring now to FIG. 6, one embodiment of a probe unit 100 is schematically depicted in which the detection unit 200 is attached to the probe unit 100. In this embodiment the various components of the detection unit 200 may be incorporated in a single integrated circuit (IC) package, for example. In this embodiment, the detection unit 200 may be attached to the distal surface 114 of the mounting member 110. In this instance, after the probe unit 100 is used on a subject, the user may discard the probe unit 100 along with the detection unit 200. That is, the probe unit 100 and the detection unit 200 are disposable. In some other embodiments, the detection unit 200 may be detachable from the probe unit 100 such that the detection unit 200 is reusable while the probe unit 100 is disposable. In this embodiment, the detection unit 200 may be enclosed in a hermetically sealed package which may be sterilized such as by autoclaving or the like. The placement of the detection unit 200 on the probe unit 100 may considerably reduce the size and footprint of the sensing apparatus 10, making at least the probe unit 100 and detection unit 200 suitable for use as a wearable device.

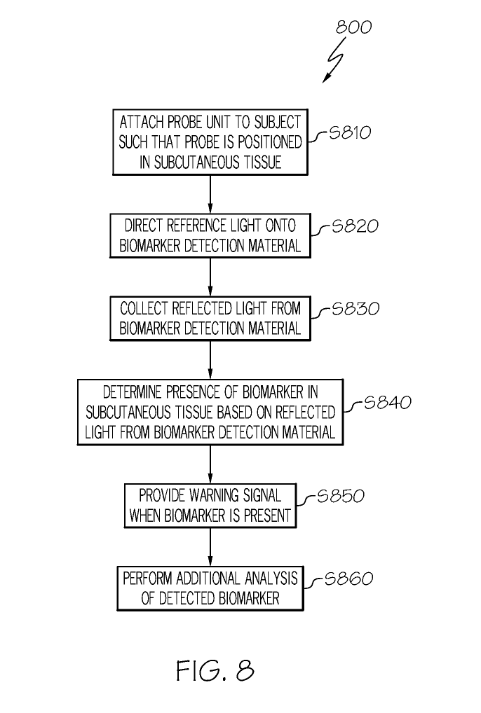

Now referring to FIGS. 1 and 7, the sensing apparatus 10 may further include a receiving unit 300 which is communicatively coupled to the detection unit 200. The receiving unit 300 is configured to receive the biomarker detection signal from the detection unit 200 and, when the biomarker detection signal indicates the presence of a biomarker, provide a warning indication, such as a visual and/or audible warning indication, to a user of the sensing apparatus 10. In some embodiments, the receiving unit 300 is configured to determine the presence of a biomarker based on the biomarker detection signal. In some embodiments, the receiving unit 300 may be configured to track and index the presence of biomarkers in the subcutaneous tissue of a subject as a function of time based on the biomarker detection signal received from the sensing unit in order to facilitate diagnosis of the development of pressure ulcers and the initiation of remedial actions.

In the embodiment of the receiving unit 300 schematically depicted in FIG. 7, the receiving unit 300 includes a receiver 310, an electronic control unit (ECU) 320, a notification unit 325, and a power source 350. The receiver 310, ECU 320, notification unit 325, and power source 350 are coupled to one another via the communication pathway 315 and the electronic control unit (ECU) 320. The various components of the receiving unit 300 are described below in further detail.

In the embodiments described herein, the communication pathway 315 may be formed from any medium that is suitable for transmitting data in the form of electrical and/or optical signals such as, for example, conductive wires, conductive traces, optical waveguides, or the like. In one exemplary embodiment, the receiver 310, ECU 320, notification unit 325, and power source 350 are positioned on an electronic substrate, such as a silicon wafer or the like, and the communication pathway 315 is a series of electrical traces interconnecting the various components of the receiving unit 300.

As noted above, the receiving unit 300 includes a receiver 310. The receiver 310 may be communicatively coupled to the ECU 320, as described herein above. In the embodiments described herein, the receiver 310 communicatively couples the receiving unit 300 to the detection unit 200, specifically to the transmitter 220 of the detection unit 200. In embodiments, the receiver 310 is communicatively coupled to the detection unit 200 wirelessly, such as when the receiver 310 is an RF receiver using Bluetooth® communication protocols, IEEE 802.11 wireless communication protocols, near-field communication protocols, or any other communication protocol suitable for facilitating radio frequency communications between electronic devices. Alternatively, the receiver 310 may be directly coupled to the detection unit 200, such a by wires and/or optical fiber.

As shown in FIG. 7, the receiving unit 300 includes an ECU 320. In embodiments, the ECU 320 may be communicatively coupled to the receiver 310, and the notification unit 325. The ECU 320 is also coupled to the power source 350 which provides electrical power to the ECU 320 and the various other components of the receiving unit 300 via the communication pathway 315. The structure of the ECU 320 is similar to the ECU 240 discussed herein above with respect to FIG. 5. That is, the ECU 320 may include a processor 324 for executing machine readable and executable instructions and a non-transitory electronic memory 322 for storing machine readable and executable instructions. In embodiments, the processor 324 may be an integrated circuit, a microchip, computer, or any other computing device capable of executing machine readable and executable instructions. The electronic memory 322 may be RAM, ROM, flash memory, a hard drive, or any other form of non-transitory memory capable of storing machine readable instructions. In the embodiments described herein, the processor 324 and the electronic memory 322 are integral with the ECU 320. However, it is noted that, in alternative embodiments, the ECU 320, the processor 324, and the electronic memory 322 may be a series of discrete components in electrical communication with one another. In embodiments, the ECU 320 may be configured to receive the biomarker detection signal from the detection unit 200 via the receiver 310 and communication pathway 315. In embodiments, the ECU 320 may be configured to analyze the biomarker detection signal and determine the presence or absence of a biomarker in the interstitial fluid of the subcutaneous tissue in which the probe unit 100 is inserted. Accordingly, it should be understood that the machine readable and executable instructions stored in the electronic memory 322 of the ECU 320 facilitate the operation of the receiving unit 300 including, without limitation, the receipt of the biomarker detection signal from the detection unit 200 and, in embodiments, the determination of the presence of a biomarker based on an analysis of the biomarker detection signal.

Still referring to FIG. 7, the receiving unit 300 further includes a notification unit 325 communicatively coupled to the ECU 320 via the communication pathway 315. In embodiments, the notification unit 325 is used to provide a user with a visual and/or audible indication of the presence of a biomarker based on the biomarker detection signal received from the detection unit 200. In embodiments, the notification unit 325 may include a display unit 330, for providing visual indications such as the presence of the at least one biomarker, absence of the at least one biomarker, characteristics of the biomarker and the like. In some embodiments, the display unit 330 includes any medium capable of transmitting an optical output such as, for example, a cathode ray tube (CRT) display, a light emitting diode (LED) display, light emitting diodes, a liquid crystal display (LCD), a plasma display, or the like. For example, in embodiments, the display unit 330 may be an LCD display or an LED display or the like, as depicted in FIG. 7. In these embodiments, the ECU 320 of the receiving unit 300 may instruct the display unit 330 to display a visual message, such as “Biomarker Detected” or the like, when a biomarker is detected in the interstitial fluid of the subcutaneous tissue in which the probe unit 100 is inserted. In other embodiments (not shown) the display unit 330 may comprise a warning light such as an LED or the like. In these embodiments, the ECU 320 of the receiving unit 300 may instruct the display unit 330 to illuminate the warning light when a biomarker is detected in the interstitial fluid of the subcutaneous tissue in which the probe unit 100 is inserted. In some embodiments, the display unit 330 may be configured to display a characteristic of the biomarker detected, such as the type of biomarker, the concentration of the biomarker in the interstitial fluid, and the like. In some embodiments, the display unit 330 may be configured to provide information regarding the location of the biomarker within the subcutaneous tissue (e.g., depth of the biomarker in the tissue).

Still referring to FIG. 7, in some embodiments the receiving unit 300 may further include an audible indicator 340 as a part of the notification unit 325. The audible indicator 340 may be used to provide an audible output related to the presence or absence of the at least one biomarker. In some embodiments, the audible indicator 340 may include any device suitable for transmitting an audible signal including, without limitation, audio speakers, mechanical or electro-mechanical transducers, or the like.

As noted hereinabove, the receiving unit 300 includes a power source 350. The power source 350 is communicatively coupled to the other components of the receiving unit 300 including the receiver 310, the notification unit 325, and the ECU 320, thereby providing electrical power to each of these components. In embodiments, the power source 350 may be an AC power source or, alternatively, a DC power source such as a battery or the like.

While FIG. 7 depicts the receiving unit 300 as being a stand-alone device, it should be understood that other configurations are contemplated and possible. For example, in embodiments, the components and/or functions of the receiving unit 300 may be incorporated in a handheld device of a user, such as a smart phone, tablet, and/or laptop computer. Alternatively, the components and/or functions of the receiving unit 300 may be incorporated in a computer, such as a desktop computer or the like, located at a nurse's or caregiver's station.

Reference will now be made to methods of using the sensing apparatus 10 with specific reference to FIGS. 1-8.

FIG. 8 is a flow chart of a method 800 for detecting a presence of at least one biomarker in subcutaneous tissue using the sensing apparatus 10 described above and shown in FIGS. 1-7. While the method 800 discusses steps to detect the presence of the at least one biomarker based on an optical characteristic of the biomarker detection material 130, it should be understood that similar steps may also be used to detect the presence of at least one biomarker based on electrical characteristics of the biomarker detection material 130. It is noted that while the method 800 depicts steps in a specific sequence, additional embodiments of the present disclosure are not limited to any particular sequence of steps.

Referring to FIG. 8, in step S810, the probe unit 100 is positioned on a subject such that the probe 120 extends into the subcutaneous tissue of the subject, as depicted in FIG. 4. The probe unit 100 may be positioned on locations of the body more prone to pressure ulcer development including, without limitation, the heels, sacrum, shoulders, and buttocks. In embodiments, positioning of the probe 120 may require angling the probe 120 with respect to the skin of the subject in such a manner that the probe 120 pierces the epidermis and dermis layers of the skin to reach the subcutaneous tissue. In embodiments, the probe 120 may be coated with a biomarker detection material 130 suitable for detecting a single biomarker in the subcutaneous tissue or a biomarker detection material suitable for detecting multiple biomarkers in the subcutaneous tissue.

The position of the probe 120 on the subject may be maintained by adhering the mounting member 110 of the probe unit 100 to the skin of the subject. For example, in embodiments, the proximal surface 112 of the mounting member 110 may be coated with an adhesive, as described herein, such that after the probe 120 is inserted into the skin of the subject, the mounting member adheres to the skin of the subject and maintains the position of the probe 120 on the subject.

At step S820, the reference light from the light source 230 of the detection unit 200 is directed onto the biomarker detection material 130 through the at least one optical fiber 128.

At step S830, the reflected light from the biomarker detection material 130 is collected with the at least one optical fiber 128 and directed to the sensor 210 of the detection unit 200 wherein an optical property of the reflected light is detected by the sensor 210. In embodiments, when the reference light is incident on the biomarker detection material 130 in the presence of the at least one biomarker, the interaction of the at least one biomarker with the biomarker detection material 130 changes an optical property of the biomarker detection material 130 which, in turn, changes an optical property of the reference light as the reference light is reflected from the biomarker detection material 130 as reflected light. This change in the optical property of the reflected light relative to the reference light is indicative of the presence of the biomarker in the interstitial fluid of the subcutaneous tissue.

In step S840, the presence of a biomarker is determined based on an optical property of the reflected light. In embodiments, the sensor 210 of the detection unit 200 outputs an electrical signal to the ECU 240 of the detection unit 200 indicative of the detected optical property of the biomarker detection material 130. This electrical signal may be indicative of, for example, a wavelength and/or an intensity of the reflected light from the biomarker detection material 130. In one embodiment, the ECU 240 of the detection unit 200 compares the optical property of the reflected light with the same optical property of the reference light to determine if a biomarker is present in the subcutaneous tissue. More specifically, the ECU 240 may contain the wavelength and/or the intensity of the reference light emitted from the light source 230 of the detection unit 200 in the electronic memory 242 of the ECU 240. The ECU 240 of the detection unit 200 compares the wavelength and/or intensity of the reference light emitted from the light source 230 to the wavelength and/or intensity of the reflected light detected by the sensor 210 as indicated by the electrical signal output from the sensor 210. When the optical property of the reflected light is the same as the optical property of the reference light, no biomarker is present in the subcutaneous tissue. However, when the ECU 240 determines that there is a difference between the optical property of the reference light and the optical property of the reflected light, a biomarker is present in the subcutaneous tissue and the ECU 240 determines a biomarker detection signal indicating that a biomarker is present. In embodiments, the presence of a biomarker may be determined based on a change in the wavelength or intensity of the reflected light relative to the reference light. The presence of the biomarker, in turn, is indicative of the development of a pressure ulcer in the subcutaneous tissue. In addition, the magnitude of the change in the wavelength or intensity of the reflected light may be indicative of the concentration of the biomarker in the tissue and, hence, may provide an indication of the rate or state of development of the pressure ulcer.

When the ECU 240 determines a biomarker detection signal based on a change in the wavelength or intensity of the reflected light, the ECU 240 transmits the biomarker detection signal to the receiving unit 300 with the transmitter 220 for further processing, storage, and analysis. In embodiments, the biomarker detection signal may include various information related to the optical property of the reflected light including, without limitation, an indication of the presence of a biomarker; the time of detection; the wavelength and/or intensity of the reflected light; the difference in the wavelength and/or intensity of the reflected light and the reference light; and/or the concentration of the biomarker. In embodiments where the detection unit 200 has a warning indicator, the ECU 240 may activate the warning indicator to provide a user with a visual or audible indication of the presence of a biomarker.

While the ECU 240 of the detection unit 200 has been described herein as comparing the optical property of the reflected light with the optical property of the reference light to determine the absence or presence of a biomarker, it should be understood that other embodiments are contemplated and possible. For example, in one embodiment, the ECU 320 of the receiving unit 300 may compare the optical property of the reflected light with the optical property of the reference light to determine the absence or presence of a biomarker. In this embodiment, the biomarker detection signal sent from the detection unit 200 to the receiving unit 300 is indicative of the optical property of the reflected light from the biomarker detection material as detected with the sensor 210 of the detection unit 200. In this embodiments, the receiving unit 300 determines the presence of a biomarker based on the biomarker detection signal sent from the detection unit 200 and, when a biomarker is present, performs additional processing, storage, and analysis on the biomarker detection signal to, for example, determine the type of biomarker present, the concentration of the biomarker, or the like.

Once the biomarker detection signal is transmitted to and received by the receiver 310 of the receiving unit 300, at step S850 the ECU 320 of the receiving unit 300 provides a warning to a user with the notification unit 325 when a biomarker is detected in the subcutaneous tissue. In embodiments, this warning may be an audible warning and/or a visual warning. This audible warning and/or visual warning may allow for a user and/or caregiver to immediately intervene and take remedial action to mitigate the development of the pressure ulcer. For example and without limitation, in response to the audible warning and/or the visual warning, the user and/or caregiver may institute steps to reposition the subject, cool the skin proximate the probe unit detecting the biomarker, and/or dry the skin proximate the probe unit detecting the biomarker.

In addition, the biomarker detection signals received from the detection unit 200 are recorded in the electronic memory 322 of the ECU 320 of the receiving unit 300 as a function of time. This continuous monitoring allows for improved detection of leading indicators of the development of pressure ulcers and, as such, allows for remedial measures to be quickly instituted to prevent the further development of pressure ulcers.

In embodiments, the notification unit 325 may provide a visual and/or audible output related to a characteristic of the detected biomarker based on the analysis of the ECU 320. In certain embodiments, the notification may be in the form of a visual display using the display unit 330. The visual display may be in the form of graphs or charts, text, or an interactive graphical user interface. In some embodiments, the notification may be in the form of an audible notification using the audible indicator 340, such as an alarm, or an automated voice reading out the name and or concentration of the at least one biomarker detected. In some embodiments, the notification unit 325 may also be configured to generate and send an email or text message regarding the presence of the at least one biomarker.

For example, in embodiments, the display unit 330 of the receiving unit 300 may display a concentration of the biomarker as a function of time. In this embodiment, the ECU 320 of the receiving unit 300 may determine the concentration of the biomarker based on the detected optical property of the reflected light from the biomarker detection material 130. In this example, the electronic memory 322 of the ECU 320 may contain a look-up table (LUT) containing concentrations of biomarkers indexed according to the change in the wavelength and/or intensity of the reference light as determined from the reflected light. Thus, the concentration of the biomarker may be determined based on the detected optical property of the reflected light and displayed as a function of time.

In other embodiments, the display unit 330 of the receiving unit 300 may display the identity of the biomarker detected. In this embodiment, the ECU 320 of the receiving unit 300 may determine the identity of the biomarker based on the detected optical property of the reflected light from the biomarker detection material 130. In this example, the electronic memory 322 of the ECU 320 may contain an LUT containing the identification of biomarkers indexed according to the change in the wavelength and/or intensity of the reference light as determined from the reflected light. Thus, the identity of the biomarker may be determined based on the detected optical property of the reflected light and displayed with the display unit 330.

In embodiments, the ECU 320 of the receiving unit 300 may optionally perform additional analysis on the biomarker detection signal at step S860. For example, in embodiments, the ECU 320 of the receiving unit 300 may determine the concentration of the biomarker based on the detected optical property of the reflected light from the biomarker detection material 130. In this example, the electronic memory 322 of the ECU 320 may contain an LUT containing concentrations of biomarkers indexed according to the change in the wavelength and/or intensity of the reference light as determined from the reflected light. Using this information, the ECU 320 may record the concentration of biomarker as function of time and store this information in the electronic memory 322 of the ECU 320. This information may then be correlated to empirical observations of wound status over time and used for historic trend review and to identify correlations between pressure ulcer development and the concentration of certain biomarkers in the subcutaneous tissue.

In other embodiments, the ECU 320 may contain an LUT containing the identity of different biomarkers indexed according to the change in the wavelength of the reference light as determined from the reflected light. Using this information, the ECU 320 of the receiving unit 300 may determine the identity of the biomarker or biomarkers present in the subcutaneous tissue and record this information as a function of time in the electronic memory 322 of the ECU 320. This information may then be correlated to empirical observations of wound status over time and used for historic trend review and to identify correlations between pressure ulcer development and the type of biomarkers in the subcutaneous tissue.

This additional analysis of the biomarker detection signal, taken in conjunction with empirical observations, may be used to refine biomarker target thresholds for specific segments of the subject population and, hence, improve the early detection and prevention of pressure ulcers. For example, in embodiments, historic data related to the type and/or concentration of biomarker may be not only indexed according to time of detection, but also to patient demographics. This indexed data may then be used to further refine biomarker target thresholds for both detection and intervention based on patient demographics.

In addition, the concentration and/or type of biomarker as a function of time may be used in a signal processing algorithm which may be applied to newly collected data to assist in the detection of future pressure ulcers.

In some embodiments of the method 800, the probe unit 100 may be discarded after removing the probe 120 from the subcutaneous tissue of the subject. Further, in certain embodiments, the probe unit 100 may be worn by the subject over extended periods of time for continuous monitoring of the presence of the at least one biomarker within the subcutaneous tissue, and therefore, the presence of the at least one biomarker may be recorded as a function of time for historical trend review.

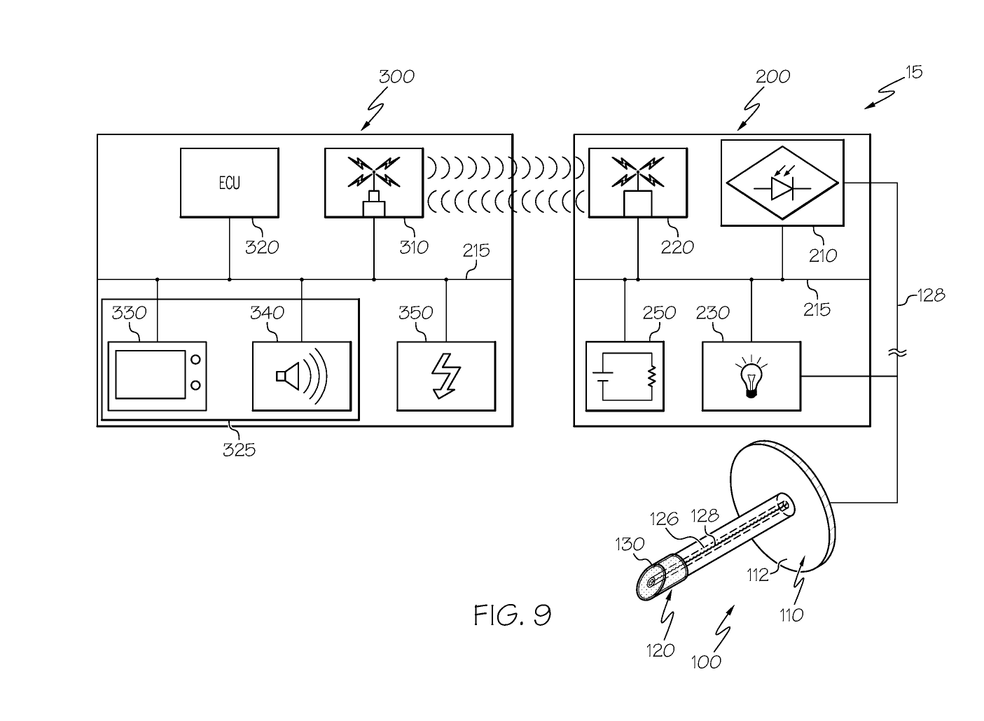

While the operation of the sensing apparatus 10 has been described herein with specific reference to FIGS. 1-7, it should be understood that other embodiments and configurations of the sensing apparatus are contemplated and possible. For example, FIGS. 9 and 10 schematically depict alternative embodiments of sensing apparatuses 15, 20, each of which are described in further detail herein.

Referring now to FIG. 9, an alternative sensing apparatus 15 for detecting at least one biomarker in the subcutaneous tissue of a subject is shown. In this embodiment of the sensing apparatus 15, the presence of the at least one biomarker is detected based on the change in an optical property of the biomarker detection material 130. As shown in FIG. 9, the sensing apparatus 15 comprises the probe unit 100, the detection unit 200, and the receiving unit 300, as described hereinabove. It is noted that while the sensing apparatus 15 is depicted in isolation, one or more components of the sensing apparatus 15 may be embedded within a mobile device (e.g. a laptop computer, a smart phone, tablet, or a wearable device) that may be carried by a user. For example, in one embodiment, the receiving unit 300 may be integrated in a smart phone or tablet of a user.

In this embodiment, the probe unit 100 includes the mounting member 110 and the probe 120, where the probe 120 comprises a shaft 124 and a lumen 126, as described hereinabove. At least one optical fiber 128 is disposed within and extends through the lumen 126. The probe 120 is coated with the biomarker detection material 130, as described herein above with respect to FIG. 2, such that a portion of the shaft 124 (and/or the lumen 126) is coated with the biomarker detection material 130. The at least one optical fiber 128 is coupled to the biomarker detection material 130.

As shown in FIG. 9, the light source 230 of the receiving unit 300 is optically coupled to the at least one optical fiber 128 and directs reference light onto the biomarker detection material 130 through the at least one optical fiber 128. In addition, the at least one optical fiber 128 may be optically coupled to the sensor 210 of the detection unit 200, thereby optically coupling the sensor 210 to the biomarker detection material 130. The sensor 210 is configured to detect an optical property of the biomarker detection material 130 from reflected light received from the biomarker detection material 130 through the at least one optical fiber 128. In embodiments, the sensor 210 may be configured to detect, for example, a wavelength or an intensity of the reflected light. In this embodiment, the sensor 210 outputs an electrical signal indicative of the detected optical property and this electrical signal is transmitted to the receiving unit 300 by the transmitter 220. Accordingly, it should be understood that, in this embodiment, the detection unit 200 does not contain a separate electronic control unit and, as such, the electrical signal output from the sensor 210 and transmitted by the transmitter 220 is the biomarker detection signal.

Still referring to FIG. 9, the biomarker detection signal transmitted by the transmitter 220 is received by the receiver 310 of the receiving unit 300 and conveyed to the ECU 320 for further analysis. In this embodiment, the ECU 320 is configured to determine the presence and/or a characteristic of at least one biomarker based on the detected optical property of the biomarker detection material 130. In embodiments, characteristics of the at least one biomarker such as the type of biomarker detected, the concentration of the biomarker detected, and/or the probability of formation of a pressure ulcer based on the detected biomarker may be determined by the ECU 320. These characteristics may be determined based on a set of predetermined parameters including, without limitation, the wavelength(s) of light reflected from the biomarker detection material, the intensity of light reflected from the biomarker detection material, and the like.

As shown in FIG. 9, the notification unit 325 is used to notify the user of the presence of the at least one biomarker when the ECU 320 determines that a biomarker is present. Additionally, the notification unit 325 may also be used to notify the user of the characteristic of the at least one biomarker as determined by the ECU 320.

Still referring to FIG. 9, in certain embodiments, when the at least one biomarker is present in the interstitial fluid of the subcutaneous tissue, the ECU 320 of the receiving unit 300 compares the optical property of the reflected light with an optical property of the reference light. In this embodiment, the optical property of the reference light may be, for example, stored in a memory of the ECU 320. In some embodiments, based on this comparison, the ECU 320 further determines a characteristic of the at least one biomarker based on the comparison of the optical property of the reflected light with the optical property of the reference light. In embodiments, the ECU 320 then provides a signal to the notification unit 325 to display a message thereon indicating the presence of the at least one biomarker, and/or the characteristic of the at least one biomarker.

In other embodiments, when no biomarker is present in the interstitial fluid of the subcutaneous tissue, the optical property of the reflected light and the optical property of the reference light are the same and, as such, the ECU 320 determines that there is no difference between the optical property of the reflected light and the optical property of the reference light. In this instance, the ECU 320 may provide a signal to the notification unit 325 indicating that no biomarker has been detected. Alternatively, the ECU 320 may not provide any signal to the notification unit 325 when the presence of a biomarker is not detected.

Now referring to FIG. 10, another alternative embodiment of a sensing apparatus 20 for detecting at least one biomarker in the subcutaneous tissue of a subject is shown. In this embodiment of the sensing apparatus 20, the presence of the at least one biomarker is detected based on the change in an electrical property of the biomarker detection material 130. Similar to the sensing apparatus 15 shown in FIG. 9, the sensing apparatus 20 also comprises the probe unit 100, the detection unit 200, and the receiving unit 300. It is noted that while the sensing apparatus 20 is depicted in isolation, one or more components of the sensing apparatus 20 may be embedded within a mobile device (e.g., a laptop computer, a smart phone, tablet, or a wearable device) that may be carried by the user. For example, in one embodiment, the receiving unit 300 may be integrated in a smart phone or tablet of a user.

Since the sensing apparatus 15 of FIG. 9 and the sensing apparatus 20 of FIG. 10 operate in a similar manner, only the differences between the two sensing apparatuses are explained herein. In the embodiment of the sensing apparatus 20 depicted in FIG. 10, the probe unit 100 comprises a mounting member 110 and a probe 120. The probe 120 includes a shaft 124 and a lumen 126 extending there through. An electrical wire 129 (instead of an optical fiber) is fitted within the lumen 126 of the probe 120. The electrical wire 129 of the probe 120 is electrically coupled to the sensor 210 and to the biomarker detection material 130. In embodiments, the electrical wire 129 is configured to pass a reference electrical signal to the biomarker detection material 130, and collect a returned electrical signal from the biomarker detection material. The reference electrical signal is sent through the biomarker detection material 130 using the electrical wire 129 by the power source 235 of the detection unit 200 (instead of using a light source 230 as shown in the embodiment of the sensing apparatus 20 depicted in FIG. 9). Further, as shown in FIG. 10 the sensor 210 is configured to determine an electrical property of the biomarker detection material 130 based on the reference signal and/or a returned electrical signal from the biomarker detection material 130. In embodiments, the sensor 210 may be configured to detect the voltage across, capacitance of, current through, or impedance of the biomarker detection material based on the returned electrical signal and/or a combination of the returned electrical signal and the reference electrical signal. In this embodiment, the sensor 210 outputs an electrical signal indicative of the detected electrical property and this electrical signal is transmitted to the receiving unit 300 by the transmitter 220. Accordingly, it should be understood that, in this embodiment, the detection unit 200 does not contain a separate electronic control unit and, as such, the electrical signal output from the sensor 210 and transmitted by the transmitter 220 is the biomarker detection signal.

In embodiments, the receiver 310 of the receiving unit 300 may receive the biomarker detection signal from the detection unit 200 and the ECU 320 may further analyze this data to determine the presence and/or a characteristic of the at least one biomarker and provide a notification to a user regarding the same in a similar manner as described hereinabove with respect to FIG. 9.

It should be understood that the embodiments disclosed herein include apparatuses and methods for detecting biomarkers in the interstitial fluid of the subcutaneous tissue of a subject based on a change in at least one of an electrical property and an optical property of a biomarker detection material associated with a probe positioned in the subcutaneous tissue of the subject. By continuously sensing and detecting the presence of certain biomarkers as they are released by cells into the interstitial fluid of the subcutaneous tissue at a specific location and in real time, early detection of pressure ulcers is possible, and the labor associated with the detection of biomarkers indicative of pressure ulcers is considerably reduced as blood samples need not be drawn at regular intervals for screening. Continuous monitoring of the interstitial fluid of the subcutaneous tissue at a specific location that is more prone to pressure ulcer formation also increases the likelihood of biomarker detection compared to techniques such as blood draw, which only detects diffuse concentrations of biomarker compounds that have gradually diffused across the dermis and epidermis and into the bloodstream. In addition, because the sensors may be positioned on areas of the body more prone to pressure ulcer development, more accurate information may be obtained about the location of developing pressure ulcers.

Moreover, the data collected from the apparatus indicative of the presence of a biomarker and/or a characteristic of the biomarker may be stored as a function of time and used for historical trend review or even as a diagnostic tool for predicting the occurrence of a pressure ulcer based on the trends. For example, based on this collected data, a signal processing algorithm may be used to predict the development of pressure ulcers based on historical trends and, based on these predictions, remedial measures may be employed to mitigate or even prevent the future occurrence of pressure ulcers. Such algorithms may be used in conjunction with individual subject or, alternatively, with certain segments of a patient population. For example, historical data of biomarker levels may be indexed according to empirical outcomes and patient profiles and, thereafter, may be used to refine target threshold levels for biomarkers within specific segments of the patient population, thereby improving pressure ulcer prediction and prevention.

It should be understood that the apparatuses and methods described herein include a number of different aspects which may be utilized in conjunction with one another in various combinations.

According to a first aspect, a sensing apparatus for detecting a development of a pressure ulcer in a subcutaneous tissue, the sensing apparatus comprising: a probe; a biomarker detection material coated on at least a portion of the probe, wherein an electrical property or an optical property of the biomarker detection material changes upon exposure to a pressure ulcer biomarker; and a sensor coupled to the biomarker detection material, the sensor configured to detect the electrical property or the optical property of the biomarker detection material, wherein a change in the electrical property or the optical property of the biomarker detection material is indicative of the development of the pressure ulcer.

In a second aspect, the sensing apparatus according to the first aspect further includes: a lumen extending through the probe; and an optical fiber positioned in the lumen of the probe and optically coupled to the biomarker detection material and the sensor, wherein the optical fiber is positioned to emit a reference light onto the biomarker detection material and collect a reflected light from the biomarker detection material.

In a third aspect, the sensing apparatus of the second aspect includes the biomarker detection material disposed in at least a portion of the lumen.

In a fourth aspect, the sensing apparatus of the second or third aspects includes a light source optically coupled to the optical fiber, the light source emitting the reference light.

A fifth aspect includes the sensing apparatus of the second through fourth aspects wherein the sensor is configured to detect at least one of a wavelength and an intensity of the reflected light.

A sixth aspect includes the sensing apparatus of the first through fifth aspects, wherein the biomarker detection material comprises: a hydrogel; and one or more components from an ELISA assay, an immuno-nephelometry assay, an immunoturbidimetry assay, rapid-enzyme immunoassays, an immunoenzymometric assay, or an immunoradiometric assay.

A seventh aspect includes the sensing apparatus of any of the first through sixth aspects, wherein the pressure ulcer biomarker comprises creatine kinase, myoglobin, H-FABP, troponin, myosin, c-reactive protein, creatinine phosphokinase, interlunkin 1-α, and/or combinations thereof.

In an eighth aspect the sensing apparatus of any of the first through seventh aspects further includes a mounting member attached to the probe, the mounting member comprising a proximal surface from which the probe extends, the proximal surface having an adhesive disposed thereon.

A ninth aspect includes the sensing apparatus of the eighth aspect wherein the sensor is positioned on a distal surface of the mounting member.

In a tenth aspect, the sensing apparatus of any of the first through ninth aspects further includes a transmitter communicatively coupled to the sensor, the transmitter transmitting a biomarker detection signal indicative of the development of the pressure ulcer.

In an eleventh aspect, the sensing apparatus of any of the first through tenth aspects further includes a receiving unit that receives the biomarker detection signal from the transmitter, the receiving unit comprising a notification unit configured to provide at least one of a visual warning and an audible warning when the biomarker detection signal is indicative of the development of the pressure ulcer.

A twelfth aspect includes the sensing apparatus of the eleventh aspect, wherein the receiving unit further comprises an electronic control unit configured to determine a characteristic of the pressure ulcer biomarker based on the biomarker detection signal and store the characteristic of the pressure ulcer biomarker in a memory of the receiving unit as a function of time.

A thirteenth aspect includes the sensing apparatus of the twelfth aspect, wherein the characteristic of the pressure ulcer biomarker is a concentration of the pressure ulcer biomarker or an identification of the pressure ulcer biomarker.

In a fourteenth aspect, the sensing apparatus of any of the first through thirteenth aspects further includes: a lumen extending through the probe; and an electrical wire positioned in the lumen of the probe and electrically coupled to the biomarker detection material, the sensor, and a power source, wherein the sensor is configured to detect the electrical property of the biomarker detection material.

A fifteenth aspect includes the sensing apparatus of the fourteenth aspect, wherein the electrical property of the biomarker detection material is at least one of an impedance, voltage, or capacitance of the biomarker detection material.

In a sixteenth aspect, a method for detecting a presence of at least one biomarker in a subcutaneous tissue includes: positioning a probe of a probe unit into the subcutaneous tissue, the probe comprising a biomarker detection material coated on at least a portion of the probe wherein an optical property or an electrical property of the biomarker detection material changes upon exposure to a pressure ulcer biomarker; detecting the optical property or the electrical property of the biomarker detection material; determining a change in the optical property or the electrical property of the biomarker detection material; and providing a warning signal when the change in the optical property or the electrical property of the biomarker detection material is indicative of a presence of the pressure ulcer biomarker.

In a seventeenth aspect, the method of the sixteenth aspect further includes determining a characteristic of the pressure ulcer biomarker based on the change in the optical property or the electrical property of the biomarker detection material.

An eighteenth aspect includes the method of the seventeenth aspect, wherein the characteristic of the pressure ulcer biomarker is a concentration of the pressure ulcer biomarker.

A nineteenth aspect includes the method of the eighteenth aspect, wherein the characteristic of the pressure ulcer biomarker is an identification of the pressure ulcer biomarker.

A twentieth aspect includes the method of any of sixteenth through nineteenth aspects, wherein detecting the optical property or the electrical property of the biomarker detection material comprises detecting the optical property of the biomarker detection material and the method further comprises: directing a reference light onto the biomarker detection material; collecting a reflected light from the biomarker detection material; and comparing an optical property of the reference light with an optical property of the reflected light to determine a change in the optical property of the biomarker detection material.

A twenty-first aspect includes the method of any of the sixteenth through twentieth aspects, wherein the probe unit comprises: a sensor; a light source; a lumen extending through the probe; and an optical fiber positioned in the lumen of the probe and optically coupled to the biomarker detection material, the sensor, and the light source, wherein the optical fiber is positioned to direct the reference light from the light source onto the biomarker detection material and direct the reflected light from the biomarker detection material to the sensor.

A twenty-second aspect includes the method of any of the sixteenth through twentieth aspects, wherein detecting the optical property or the electrical property of the biomarker detection material comprises detecting the electrical property of the biomarker detection material and the probe unit comprises: a sensor; a power source; a lumen extending through the probe; and an electrical wire positioned in the lumen of the probe and electrically coupled to the biomarker detection material, the sensor, and the power source, the sensor detecting the electrical property of the biomarker detection material through the electrical wire.

A twenty-third aspect includes the method of the twenty-second aspect, wherein the electrical property of the biomarker detection material is at least one of an impedance, voltage, or capacitance of the biomarker detection material.

A twenty-fourth aspect includes the method of any of the sixteenth through twenty-third aspects, wherein the biomarker detection material comprises one or more components from an ELISA assay, an immuno-nephelometry assay, an immunoturbidimetry assay, rapid-enzyme immunoassays, an immunoenzymometric assay, or an immunoradiometric assay.

A twenty-fifth aspect includes the method of any of the sixteenth through twenty-fourth aspects, wherein the pressure ulcer biomarker comprises creatine kinase, myoglobin, H-FABP, troponin, myosin, c-reactive protein, creatinine phosphokinase, interlunkin 1-α, and/or combinations thereof.

A twenty-sixth aspect includes a sensing apparatus for detecting a development of a pressure ulcer in a subcutaneous tissue, the sensing apparatus comprising: a probe unit comprising a probe having a biomarker detection material coated on at least a portion of the probe, wherein an optical property of the biomarker detection material changes upon exposure to a pressure ulcer biomarker; a detection unit comprising a sensor and a light source emitting a reference light; and at least one optical fiber optically coupling the light source to the biomarker detection material and optically coupling the sensor to the biomarker detection material, wherein: the sensor is configured to detect an optical property of the biomarker detection material, a change in the optical property of the biomarker detection material being indicative of the development of the pressure ulcer.

A twenty-seventh aspect includes the sensing apparatus of the twenty-sixth aspect, wherein the detection unit further comprises: a transmitter; and an electronic control unit communicatively coupled to the transmitter and the sensor, the electronic control unit comprising a processor and a memory storing machine readable and executable instructions which, when executed by the processor: determine a biomarker detection signal based on the optical property of the biomarker detection material; and transmit the biomarker detection signal with the transmitter.

A twenty-eighth aspect includes the sensing apparatus of any of the twenty-sixth or twenty seventh aspects, wherein the machine readable and executable instructions of the detection unit, when executed by the processor, cause the electronic control unit of the detection unit to: compare an optical property of the reference light emitted by the light source with an optical property of a reflected light received by the sensor from the biomarker detection material, wherein the biomarker detection signal is based on the comparison.

A twenty-ninth aspect includes the sensing apparatus of any of the twenty-sixth through twenty-eighth aspects, wherein: the detection unit further comprises a warning indicator communicatively coupled to the electronic control unit of the detection unit; and the machine readable and executable instructions of the detection unit, when executed by the processor, cause the electronic control unit of the detection unit to activate the warning indicator when the biomarker detection signal is indicative of the presence of a pressure ulcer biomarker.

In a thirtieth aspect, the sensing apparatus of any of the twenty-sixth through twenty-ninth aspects, further comprising a receiving unit, the receiving unit comprising: a receiver; a notification unit; and an electronic control unit communicatively coupled to the receiver and the notification unit, the electronic control unit of the receiving unit comprising a processor and a memory storing machine readable and executable instructions which, when executed by the processor: cause the electronic control unit of the receiving unit to receive the biomarker detection signal from the detection unit; determine if a pressure ulcer biomarker is present based on the biomarker detection signal; and provide a warning with the notification unit when the pressure ulcer biomarker is present.

A thirty-first aspect includes, the sensing apparatus of the thirtieth aspect, wherein the machine readable and executable instructions of the receiving unit further cause the electronic control unit of the receiving unit to record the biomarker detection signal as a function of time in the memory of the receiving unit.

A thirty-second aspect includes, the sensing apparatus of the thirtieth or thirty-first aspects, wherein the machine readable and executable instructions of the receiving unit further cause the electronic control unit of the receiving unit to determine a characteristic of the pressure ulcer biomarker based on the biomarker detection signal.

A thirty-third aspect includes the sensing apparatus of the thirty-second aspect, wherein the machine readable and executable instructions of the receiving unit further cause the electronic control unit of the receiving unit to display the characteristic of the pressure ulcer biomarker with the notification unit.

A thirty-fourth aspect includes the sensing apparatus of any of the thirtieth through thirty-third aspects, wherein the characteristic of the pressure ulcer biomarker is an identity of the pressure ulcer biomarker.

A thirty-fifth aspect includes the sensing apparatus of any of the thirtieth through thirty-third aspects, wherein the characteristic of the pressure ulcer biomarker is a concentration of the pressure ulcer biomarker.

A thirty-sixth aspect includes the sensing apparatus of any of the twenty-sixth through thirty-fifth aspects, wherein: the probe unit comprises a mounting member; the probe extends from a proximal surface of the mounting member; and the detection unit is positioned on a distal surface of the mounting member.

A thirty-seventh aspect includes the sensing apparatus of the thirty-sixth aspect, wherein the proximal surface of the mounting member comprises an adhesive.

A thirty-eighth aspect includes the sensing apparatus of any of the twenty-sixth through thirty-seventh aspects, wherein the biomarker detection material is disposed in at least a portion of a lumen of the probe.

A thirty-ninth aspect includes the sensing apparatus of any of the twenty-sixth through thirty-eighth aspects, wherein the probe comprises a lumen and the at least one optical fiber is positioned in the lumen.

A fortieth aspect includes, the sensing apparatus of any of the twenty-sixth through thirty-ninth aspects, wherein the optical property of the biomarker detection material is at least one of a wavelength and an intensity of reflected light from the biomarker detection material.

A forty-first aspect includes the sensing apparatus of any of the twenty-sixth through fortieth aspects, wherein the biomarker detection material comprises one or more components from an ELISA assay, an immuno-nephelometry assay, an immunoturbidimetry assay, rapid-enzyme immunoassays, an immunoenzymometric assay, or an immunoradiometric assay.

A forty-second aspect includes, the sensing apparatus of any of the twenty-sixth through fortieth aspects, wherein the pressure ulcer biomarker comprises creatine kinase, myoglobin, H-FABP, troponin, myosin, c-reactive protein, creatinine phosphokinase, interlunkin 1-α, and/or combinations thereof.

While particular embodiments have been illustrated and described herein, it should be understood that various other changes and modifications may be made without departing from the spirit and scope of the claimed subject matter. Moreover, although various aspects of the claimed subject matter have been described herein, such aspects need not be utilized in combination. It is therefore intended that the appended claims cover all such changes and modifications that are within the scope of the claimed subject matter.

What is claimed is:

1. A sensing apparatus for detecting a development of a pressure ulcer in a subcutaneous tissue, the sensing apparatus comprising:
a probe;
a biomarker detection material coated on at least a portion of the probe, wherein an electrical property or an optical property of the biomarker detection material changes upon exposure to a pressure ulcer biomarker; and
a sensor coupled to the biomarker detection material, the sensor configured to detect the electrical property or the optical property of the biomarker detection material, wherein a change in the electrical property or the optical property of the biomarker detection material is indicative of the development of the pressure ulcer.

2. The sensing apparatus of claim 1, further comprising:
a lumen extending through the probe; and
an optical fiber positioned in the lumen of the probe and optically coupled to the biomarker detection material and the sensor, wherein the optical fiber is positioned to emit a reference light onto the biomarker detection material and collect a reflected light from the biomarker detection material.

3. The sensing apparatus of claim 2, wherein the biomarker detection material is disposed in at least a portion of the lumen.

4. The sensing apparatus of claim 2, further comprising a light source optically coupled to the optical fiber, the light source emitting the reference light.

5. The sensing apparatus of claim 2, wherein the sensor is configured to detect at least one of a wavelength and an intensity of the reflected light.

6. The sensing apparatus of claim 1, wherein the biomarker detection material comprises:
a hydrogel; and
one or more components from an ELISA assay, an immuno-nephelometry assay, an immunoturbidimetry assay, rapid-enzyme immunoassays, an immunoenzymometric assay, or an immunoradiometric assay.

7. The sensing apparatus of claim 1, wherein the pressure ulcer biomarker comprises creatine kinase, myoglobin, H-FABP, troponin, myosin, c-reactive protein, creatinine phosphokinase, interlunkin 1-α, and/or combinations thereof.

8. The sensing apparatus of claim 1, further comprising a mounting member attached to the probe, the mounting member comprising a proximal surface from which the probe extends, the proximal surface having an adhesive disposed thereon.

9. The sensing apparatus of claim 8, wherein the sensor is positioned on a distal surface of the mounting member.

10. The sensing apparatus of claim 1, further comprising a transmitter communicatively coupled to the sensor, the transmitter transmitting a biomarker detection signal indicative of the development of the pressure ulcer.

11. The sensing apparatus of claim 10, further comprising a receiving unit that receives the biomarker detection signal from the transmitter, the receiving unit comprising a notification unit configured to provide at least one of a visual warning and an audible warning when the biomarker detection signal is indicative of the development of the pressure ulcer.

12. The sensing apparatus of claim 11, wherein the receiving unit further comprises an electronic control unit configured to determine a characteristic of the pressure ulcer biomarker based on the biomarker detection signal and store the characteristic of the pressure ulcer biomarker in a memory of the receiving unit as a function of time.

13. The sensing apparatus of claim 12, wherein the characteristic of the pressure ulcer biomarker is a concentration of the pressure ulcer biomarker or an identification of the pressure ulcer biomarker.

14. The sensing apparatus of claim 1, further comprising:
a lumen extending through the probe; and
an electrical wire positioned in the lumen of the probe and electrically coupled to the biomarker detection material, the sensor, and a power source, wherein the sensor is configured to detect the electrical property of the biomarker detection material.

15. The sensing apparatus of claim 14, wherein the electrical property of the biomarker detection material is at least one of an impedance, voltage, or capacitance of the biomarker detection material.

16. A method for detecting a presence of at least one biomarker in a subcutaneous tissue, the method comprising:
positioning a probe of a probe unit into the subcutaneous tissue, the probe comprising a biomarker detection material coated on at least a portion of the probe wherein an optical property or an electrical property of the biomarker detection material changes upon exposure to a pressure ulcer biomarker;
detecting the optical property or the electrical property of the biomarker detection material;
determining a change in the optical property or the electrical property of the biomarker detection material; and
providing a warning signal when the change in the optical property or the electrical property of the biomarker detection material is indicative of a presence of the pressure ulcer biomarker.

17. The method of claim 16 further comprising determining a characteristic of the pressure ulcer biomarker based on the change in the optical property or the electrical property of the biomarker detection material.

18. The method of claim 17, wherein the characteristic of the pressure ulcer biomarker is a concentration of the pressure ulcer biomarker.

19. The method of claim 17, wherein the characteristic of the pressure ulcer biomarker is an identification of the pressure ulcer biomarker.

20. The method of claim 16, wherein detecting the optical property or the electrical property of the biomarker detection material comprises detecting the optical property of the biomarker detection material and the method further comprises:
directing a reference light onto the biomarker detection material;
collecting a reflected light from the biomarker detection material; and
comparing an optical property of the reference light with an optical property of the reflected light to determine a change in the optical property of the biomarker detection material.

21. The method of claim 20, wherein the probe unit comprises:
a sensor;
a light source;
a lumen extending through the probe; and
an optical fiber positioned in the lumen of the probe and optically coupled to the biomarker detection material, the sensor, and the light source, wherein the optical fiber is positioned to direct the reference light from the light source onto the biomarker detection material and direct the reflected light from the biomarker detection material to the sensor.

22. The method of claim 16, wherein detecting the optical property or the electrical property of the biomarker detection material comprises detecting the electrical property of the biomarker detection material and the probe unit comprises:
a sensor;
a power source;
a lumen extending through the probe; and
an electrical wire positioned in the lumen of the probe and electrically coupled to the biomarker detection material, the sensor, and the power source, the sensor detecting the electrical property of the biomarker detection material through the electrical wire.

23. The method of claim 22, wherein the electrical property of the biomarker detection material is at least one of an impedance, voltage, or capacitance of the biomarker detection material.

24. The method of claim 16, wherein the biomarker detection material comprises one or more components from an ELISA assay, an immuno-nephelometry assay, an immunoturbidimetry assay, rapid-enzyme immunoassays, an immunoenzymometric assay, or an immunoradiometric assay.

25. The method of claim 16, wherein the pressure ulcer biomarker comprises creatine kinase, myoglobin, H-FABP, troponin, myosin, c-reactive protein, creatinine phosphokinase, interleukin 1-α, and/or combinations thereof.
